(19) United States
(12) Patent Application Publication
(10) Pub. No.: US 2006/0084659 A1
(43) Pub. Date: Apr. 20, 2006

(54) Augmentation of psychotherapy with cannabinoid reuptake inhibitors

(76) Inventors: Michael Davis, Stone Mountain, GA (US); Kerry J. Ressler, Atlanta, GA (US); Jasmeer P. Chhatwal, Atlanta, GA (US); Jason P. McDevitt, Williamsburg, VA (US)

Correspondence Address: JASON P. MCDEVITT, 124 COUNTRY CLUB DRIVE, WILLIAMSBURG, VA, 23188, US

(21) Appl. No.: 11/253,314

(22) Filed: Oct. 19, 2005

Related U.S. Application Data
(60) Provisional application No. 60/620,011, filed on Oct. 19, 2004.

(51) Int. Cl. A61K 31/519; A61K 31/195

(52) U.S. Cl. 514/252.16; 514/262.1; 514/283; 514/561

(57) ABSTRACT

Methods are provided for facilitating psychological extinction of a deleterious, high-anxiety response that is disproportionate to the threat offered by a given stimulus. An afflicted subject is treated with a cannabinoid reuptake inhibitor in conjunction with extinction training. The methods are relevant for treatment of anxiety disorders, including phobic disorders and PTSD, in addition to other afflictions such as chronic pain, insomnia, and erectile dysfunction.

### CROSS-REFERENCE TO RELATED APPLICATIONS

This application claims the benefit of priority to U.S. Provisional Patent Application 60/620,011, filed Oct. 19, 2004, the contents of which are herein incorporated by reference in their entirety.

### STATEMENT REGARDING FEDERALLY SPONSORED RESEARCH OR DEVELOPMENT

This invention was made with U.S. Government support under grants MH69884, MH47840, and MH59906 awarded by the National Institutes of Health (MD), and Agreement IBN-987675, awarded by the Science and Technology Center Program (Center for Behavioral Neuroscience) of the National Science Foundation. The U.S. Government may have certain rights in this invention.

### BACKGROUND OF THE INVENTION

The invention relates to the treatment of medical disorders by facilitating psychological extinction of high-anxiety responses to non-threatening stimuli.

Classical fear conditioning occurs when an affectively neutral stimulus is paired with a noxious aversive stimulus (unconditioned stimulus [US]) such as footshock. Afterward, the previously neutral stimulus (i.e., now the conditioned stimulus [CS]) is able to elicit a variety of autonomic, hormonal, and skeletal responses that accompany the conscious experience of fear in humans and which are used to operationally define fear in laboratory animals. The fear-eliciting properties of the CS can be extinguished by repeatedly presenting the CS in the absence of the US. It is generally believed that extinction does not reflect unlearning of the original association but involves instead the formation of new associations that compete with the previously conditioned response.

Recent studies have implicated the cannabinoid system in the learned inhibition of fear (extinction). Cannabinoid Receptor Type 1 (CB1) is densely expressed in regions known to be important for anxiety and emotional learning, including the amygdala, hippocampus, and throughout the mesolimbic dopamine reward system (Katona et al. (1999) J. Neurosci. 19(11): 4544-58; Freund et al. (2003) Physiol. Rev. 83(3):1017-66). Deletion of the gene for the CB1 cannabinoid receptor in knockout mice leads to increased anxiety and profound deficits in extinction, while the acquisition of the initial fear response remains normal (Haller et al. (2002) Eur. J Neurosci. 16(7): 1395-8; Marsicano et al. (2002) Nature 418: 530-534). Pharmacologic blockade of the CB1 receptor leads to a similar deficit in extinction in mice, further demonstrating the importance of CB1 receptor activation to extinction in mice (Marsicano et al. (2002) Nature 418: 530-534).

FAAH inhibitors have been proposed as potential therapeutics for treatment of a wide variety of clinical indications including anxiety disorders, neuropathic pain, acute pain, chronic pain, emesis, anxiety, feeding behavior, movement disorders, glaucoma, sleep disorders, brain injury, and cardiovascular disease (U.S. Pat. No. 6,699,682, and U.S. Patent Application Nos. 20040127518, 20050131032, 20030092734, 20020188009).

In addition to the cannabinoid system, N-methyl-D-aspartate (NMDA) receptor antagonists have been shown to block extinction when administered either systemically or infused directly into the amygdala (as reviewed in Davis et al. (2005), Current Directions in Psychological Science, 14(4): 214-219). In pending U.S. Patent Application No. 20050096396, Davis et al. describe use of the partial NMDA receptor agonist D-cycloserine (DCS) to facilitate extinction in rats, and subsequently in humans to facilitate extinction in conjunction with psychotherapy for treatment of phobic disorders.

A reduced ability to extinguish high-anxiety responses resulting from fear memories is a significant clinical problem for a wide range of anxiety disorders including specific phobias, panic disorder, and post-traumatic stress disorder. These disorders are characterized by a high-anxiety response to a stimulus that is highly disproportionate to the threat. Treatment for these disorders often relies upon the progressive extinction of the high-anxiety response to the stimulus, and hence pharmacological enhancement of extinction could be of considerable clinical benefit in these conditions.

A reduced ability to extinguish deleterious, high-anxiety responses also contributes to recurring medical afflictions such as erectile dysfunction, insomnia, and chronic pain. While these afflictions have widely varying etiologies and symptoms, they share a common feature, which is that their severity and frequency of the afflictions can be exacerbated by anxiety regarding the affliction. For example, an episode of impotence in a male may generate significant anxiety about the condition, which may contribute to future episodes of impotence. Approved drugs for recurrent conditions such as insomnia and erectile dysfunction target the physiology of the symptoms, but neglect the mental component of the disorder.

### BRIEF SUMMARY OF THE INVENTION

Methods are provided for facilitating, in a mammalian subject, extinction of deleterious, high-anxiety responses to stimuli that are non-aversive to most individuals. The methods comprise administering a cannabinoid reuptake inhibitor to a subject in conjunction with extinction training. The extinction training is designed to develop a new, non-deleterious response to a given stimulus that previously generated disproportionate anxiety, i.e., to extinguish the high-anxiety response by replacing it with a more appropriate response. The cannabinoid reuptake inhibitor facilitates extinction, and thus speeds up the process, thereby improving the therapeutic treatment. Pharmacologic agents that act only as agonists of cannabinoid receptors, and do not inhibit cannabinoid reuptake, will not facilitate extinction and thus are not contemplated by the methods of the invention. The methods of the invention also comprise administering to an afflicted individual a cannabinoid reuptake inhibitor and an additional pharmacologic agent that facilitates extinction by enhancing NMDA receptor activation or transmission, again in conjunction with extinction training. For example, DCS can be co-administered with a cannabinoid reuptake inhibitor to facilitate the extinction process. The methods are useful for treating a variety of afflictions for which extinction of deleterious anxiety responses would be beneficial, including anxiety disorders, addictive disorders, mood disorders, movement disorders, erectile dysfunction, chronic pain, and insomnia.

### BRIEF DESCRIPTION OF THE DRAWINGS

FIG. 1 shows expression patterns of the CB1-receptor following in situ hybridization with ³⁵S-labeled antisense riboprobe. (A) Dense CB1 expression within amygdala (arrow) and hypothalamus, with more sparse cellular expression throughout hippocampus and cortex. (B) Cresyl violet stained sections of the temporal lobe. (C) CB1 is most densely expressed within the basolateral amygdala (BLA, arrows). CeA=Central amygdaloid nucleus, MeA=Medial amygdaloid nucleus.

FIG. 2 shows a parametric evaluation of different amounts of extinction training. A graph depicts fear-potentiated startle (FPS) as a function of the extent of extinction training (lights without shocks).

FIG. 3 shows a dose-response function for the effect of SR 141716A on extinction. Percent FPS is shown for the pre-extinction test and post-extinction tests of four groups of animals that received SR 141716A (0, 0.15, 1.5, 5 mg/kg, i.p.) prior to extinction training (n=16 for 0, 1.5, and 5 mg/kg groups; n=8 for 0.15 mg/kg group) (* denotes p<0.05, ** denotes p<0.01).

FIG. 4 shows the effect of WIN 55,212-2 on extinction. FPS is graphed as a function of the presence of the CB1 agonist, WIN 55,212-2 (n=5 per group).

FIG. 5 shows the effect of AM404 on extinction. (A) Percent FPS during post-extinction testing, 24 hrs after animals received 0, 2, or 10 mg/kg AM404, i.p., prior to extinction training (n=21 for 0 and 2 mg/kg; n=29 for 10 mg/kg). (B) Percent FPS during post-extinction testing 1 hr after animals received 0, 2, or 10 mg/kg AM404, i.p., prior to extinction training (n=13 for 0 and 10 mg/kg, n=12 for 2 mg/kg).

FIG. 6 shows a comparison of AM404, rimonabant, and AM404 plus rimonabant treatment on extinction. Groups were administered 10 mg/kg AM404, 10 mg/kg AM404+5 mg/kg rimonabant, or 5 mg/kg rimonabant alone, respectively, prior to extinction training (30 trial extinction).

FIG. 7 shows percent FPS in fear conditioned animals without cue re-exposure. Fear-conditioned animals were administered AM404 (10 mg/kg), rimonabant (SR 141716A, 5 mg/kg), or vehicle, and 1 hour later were tested for FPS (cue re-exposure was omitted, n=8 per group).

FIG. 8 shows that the effect of AM404 on extinction is independent of effects on the expression of conditioned fear, pain, locomotion, and baseline anxiety. (A) Average shock reactivity is shown in arbitrary units and represents the average response to 3 footshocks. (B) Average baseline activity level is shown in arbitrary units as determined by mean displacement of accelerometers during the 2 minutes prior to the delivery of any shocks in the test chambers. (C) Average baseline startle amplitude shown in arbitrary units during the presentations of startle stimuli (n=8 per group for 8A-C).

FIG. 9 shows the effect of AM404 on shock-induced reinstatement of fear. (A) Percent FPS is shown during testing following reinstatement with 3 footshocks. Animals received vehicle (n=21) or AM404 (2-mg and 10-mg groups combined, n=42) prior to extinction training (i.e., 48 hrs prior to reinstatement)(* denotes p<0.05). (B) Within-session extinction is shown for the first 4 trials during the testing of FPS following the reinstatement experiment described in (A).

FIG. 10 shows the effect of URB597 on extinction. (A) FIG. 10A shows the ratio of FPS relative to baseline FPS for control and treated animals. (B) FIG. 10B follows progressive FPS levels within a given extinction training session for control and treated animals.

### DETAILED DESCRIPTION OF THE INVENTION

The present invention is directed to methods for facilitating extinction of a deleterious, high-anxiety response to a psychological stimulus. The methods comprise administering to a subject a therapeutically effective amount of a cannabinoid reuptake inhibitor in conjunction with extinction training. The methods also include a combination therapy protocol comprising administering to a subject a therapeutically effective amount of both a cannabinoid reuptake inhibitor and an additional pharmacologic agent, preferably an agent that enhances NMDA receptor neurotransmission, in conjunction with a session of psychotherapy.

As used herein, each of the following terms has the meaning associated with it as described below.

The articles “a” and “an” are used herein to refer to one or to more than one (i.e. to at least one) of the grammatical object of the article. By way of example, “an element” means one element or more than one element.

As used herein, “plurality” means at least two.

As used herein, “FDA” means the United States Food and Drug Administration.

Any ranges cited herein are inclusive, e.g., “between about 50 mg and 100 mg” includes compositions of 50 mg and 100 mg.

As used herein, “acute” administration of a therapeutic means a single exposure within an extended time period of the subject to the therapeutically effective amount of the pharmacologic agent that facilitates extinction. In conjunction with this definition of “acute”, an extended time period is defined as four days or longer, e.g., once-weekly administration of a cannabinoid reuptake inhibitor constitutes acute administration. Administering a dose of a cannabinoid reuptake inhibitor to a subject, followed by a second dose 24 hours later, does not constitute acute dosing. Administering a single dose of a cannabinoid reuptake inhibitor, wherein the dose is formulated to have both immediate release and delayed release characteristics, constitutes acute dosing provided that the peak blood level of the cannabinoid reuptake inhibitor in the subject is achieved within 12 hours of the time the dose is administered.

As used herein, a subject is “treated”, or subjected to “treatment”, when an earnest attempt is made to alleviate a medical disorder or disease. For example, a subject can be treated for a disorder by being administered a pharmacologic agent that is intended to alleviate the disorder, irrespective of whether the treatment actually was successful in alleviating the disorder.

As used herein, a disease or disorder or medical affliction is “alleviated” if either (or both) the severity or frequency of a symptom of the disease or disorder or medical affliction is reduced.

A “subject” of diagnosis or treatment is a mammal.

A “therapeutic” treatment is a treatment administered to a subject who exhibits signs of pathology for the purpose of diminishing or eliminating those signs.

A “therapeutically effective amount” or “therapeutically effective dose” of the pharmacologic agent is an amount of the pharmacologic agent that, when administered in conjunction with extinction training, results in an improved therapeutic benefit relative to that observed with extinction training in the absence of administering the pharmacologic agent.

As used herein, a “deleterious, high-anxiety response” refers to a subject's response to a given stimulus, wherein the response is characterized by a high level of anxiety that is disproportionate to the threat represented by the stimulus. Accordingly, a stimulus that generates little if any anxiety in most subjects would generate substantial anxiety in a subject undergoing a deleterious, high-anxiety response. These deleterious, high-anxiety responses cause or exacerbate symptoms characteristic of the medical disorders described herein.

As used herein, a pharmacologic agent that “hastens the rate of extinction” refers to a compound that, when administered to rats according to the experimental procedures described herein, significantly reduces the extent of fear-potentiated startle in treated rats (relative to untreated animals) in response to a conditioned stimulus.

As used herein, the term “neuropathic pain” means pain that originates from a damaged or malfunctioning nerve or nervous system. “Chronic pain” means pain that has lasted for more than three months, generally resulting in significant psychological and emotional affects and limiting a person's ability to fully function.

As used herein, “insomnia” is defined as the inability to fall asleep or to stay asleep for a sufficient amount of time during regular sleeping hours. It includes acute insomnia, which occurs in either a transient or short term form, and chronic insomnia. It also includes initial insomnia, defined as difficulty in falling asleep; middle insomnia, defined as awakening in the middle of the night followed by eventually falling back to sleep, but with difficulty; and terminal insomnia, defined as awakening before one's usual waking time and being unable to return to sleep.

As used herein, “biofeedback” refers to a technique in which subjects are trained to improve their health by using signals from their own bodies to control their own physiological responses. Biofeedback is particularly useful in enabling subjects to learn to control physiological processes that normally occur involuntarily, such as blood pressure, heart rate, muscle tension, and skin temperature.

As used herein, “erectile dysfunction” is impotence resulting from a man's inability to obtain or maintain an erection of his penis.

As used herein, the term “NMDA receptor” or “NMDA channel” refers to the glutamate receptor channel NMDA subtype (Yamakura and Shimoji (1999) Prog. Neurobiol. 59(3):279-298).

The term “agonist” generally refers to a compound that interacts with a receptor and initiates or facilitates a response characteristic of that receptor. The term “antagonist” generally refers to a compound that interacts with a receptor and initiates or facilitates a response counter to the natural characteristic of the receptor. The term “partial agonist” refers to a compound that regulates an allosteric site on an ionotropic receptor, such as the NMDA receptor, to increase or decrease the flux of cations through the ligand-gated channel depending on the presence or absence of the principal site ligand; i.e., in the presence or absence of a known endogenous ligand binding to a site on the receptor. In the absence of the principal site ligand, a partial agonist increases the flow of cations through the ligand-gated channel, but at a lower flux than achieved by the principal site ligand. A partial agonist partially opens the receptor channel. In the presence of the principal site ligand, a partial agonist decreases the flow of cations through the ligand-gated channel below the flux normally achieved by the principal site ligand. As used herein, “NMDA receptor agonist,” “NMDA receptor antagonist,” and “NMDA receptor partial agonist,” may be alternately referred to as “NMDA agonist,” “NMDA antagonist,” and “NMDA partial antagonist,” respectively. Also, “NMDA receptor partial agonist” is intended to be interchangeable with “partial NMDA receptor agonist.” The present invention contemplates a variety of molecules acting as such partial NMDA receptor agonists. Examples of such pharmacologic agents include, but are not limited to, compounds that act at the glycine modulatory site of the NMDA receptor, including DCS, D-serine, and 1-aminocyclopropane-carboxylic acid (ACPC) (see U.S. Pat. Nos. 5,086,072 and 5,428,069, and U.S. Patent Application No. 20050143314). NMDA receptor partial agonists are compounds that can enhance learning, and are particularly useful when used in accordance with the methods and compositions of the present invention.

As used herein, “anxiety disorder” refers to a disorder characterized by fear, anxiety, addiction, and the like that can be treated with the methods of the invention. An individual who may benefit from the methods of the invention may have a single disorder, or may have a constellation of disorders. The anxiety disorders contemplated in the present invention include, but are not limited to, fear and anxiety disorders, addictive disorders including substance-abuse disorders, and mood disorders. Fear and anxiety disorders include, but are not limited to, panic disorder, specific phobia, post-traumatic stress disorder (PTSD), obsessive-compulsive disorder, and movement disorders such as Tourette's syndrome. The disorders contemplated herein are defined in, for example, the DSM-IV (Diagnostic and Statistical Manual of Mental Disorders (4th ed., American Psychiatric Association, Washington D.C., 1994)).

“Pharmacologic agent” refers to a compound, mixture, etc., exhibiting properties indicating usefulness in a medicament.

As used herein, two pharmacologic agents are said to be “co-administered” when the pharmacologic agents are administered simultaneously in a single dosage form, or administered separately within a limited period of time of about three hours.

“Cannabinoid receptor” refers to a receptor in the endocannabinoid (EC) system, including Cannabinoid Receptor Type 1 (CB1) and Cannabinoid Receptor Type 2 (CB2) (Matsuda et al. (1990) Nature 346: 561; Munro et al. (1993) Nature 365: 61).

The term “cannabinoid reuptake inhibitor” encompasses compounds that decrease the reuptake of endogenous cannabinoids into neurons or decrease the enzymatic breakdown of endogenous cannabinoids in extracellular space, including synaptic clefts. Furthermore, as defined herein, cannabinoid reuptake inhibitors are strong inhibitors of fatty acid amide hydrolase, with half-maximal inhibitory concentrations (IC₅₀)<5 μM, as measured using a radiolabeled anandamide assay described by Mor (Mor et al., (2004) J. Med. Chem. 47(21): 4998-5008).

The term “cannabinoid receptor agonist” encompasses any compound that binds to or associates with cannabinoid receptors and initiates intracellular signaling pathways associated with cannabinoid receptors, including, for example, inhibition of adenylate cyclase, inhibition of N- and Q-type voltage-dependent calcium channels, and stimulation of an inwardly rectifying potassium current (Kᵢᵣ current). The term “cannabinoid receptor antagonist” encompasses any compound that binds to or associates with cannabinoid receptors and blocks the initiation of intracellular signaling pathways associated with cannabinoid receptors, including, for example, inhibition of adenylate cyclase, inhibition of N- and Q-type voltage-dependent calcium channels, and stimulation of an inwardly rectifying potassium current (Kᵢᵣ current). As used herein, “cannabinoid receptor agonist” and “cannabinoid receptor antagonist,” may be alternately referred to as “cannabinoid agonist” and “cannabinoid antagonist,” respectively.

“Extinction training” refers to a method wherein a subject having deleterious, high-anxiety responses to a given stimulus, is exposed to the stimulus such that the conditions of the exposure are manipulated to control the outcome or otherwise reduce the likelihood of an event occurring that would tend to reinforce the fear response. The goal of extinction training is to pair the previously aversive stimulus with a new learning resulting from a non-deleterious outcome resulting from the stimuluse, thereby generating in future exposures to the stimulus a more appropriate response in place of the previous deleterious, high-anxiety response. For example, the conditions of the exposure can be manipulated by psychotherapy or pharmacotherapy. In one example of extinction training, a subject having a phobic disorder undergoes extinction training by participating in a traditional exposure-based psychotherapy session. As another example, a subject having erectile dysfunction undergoes extinction training by taking a drug that treats the symptoms of erectile dysfunction (e.g., sildenafil) prior to engaging in a sexual interlude.

“Psychotherapy” refers to a treatment of mental illness, anxiety disorders or emotional disturbances primarily by verbal or non-verbal communication.

Administration of Cannabinoid Reuptake Inhibitors

The present invention contemplates a variety of molecules acting as cannabinoid reuptake inhibitors, including but not limited to amines as described in US Patent Application 2004/0048907, specifically including the compounds N-(4-hydroxyphenyl)arachidonamide (AM404), N-(5Z, 8Z, 11Z, 14Z eicosatetraenyl)-4-hydroxybenzamide (AM1172), and N-(2-methyl-4-hydroxy-phenyl)-arachidonamide (VDM11); the compound cyclohexylcarbamic acid 3′-carbamoylbiphenyl-3-yl ester (URB-597, described in Mor et al., (2004) J. Med. Chem. 47(21): 4998-5008); as well as fatty acid amide hydrolase (FAAH) inhibitors such as those described in US Patent Application Nos. 20040127518, 20050131032, 20030092734, and 20020188009, and literature publications (Beltramo et al. (1997) Science 277: 1094-1097; Fegley et al. (2004) Proc. Natl. Acad. Sci. USA 101(23): 8756-61; Du et al. (2005) Bioorganic & Medicinal Chemistry Letters 15(1): 103-106; Boger et al., (2005) J. Med. Chem. 48(6): 1849-1856).

In some embodiments of the invention, the cannabinoid reuptake inhibitor is co-administered with a second pharmacologic agent that facilitates extinction by a different mechanism. In some embodiments, DCS is co-administered with a cannabinoid reuptake inhibitor. DCS has been FDA-approved for approximately 20 years for the treatment of tuberculosis. It has been tested as a cognitive enhancer in several clinical trials over the last decade. For tuberculosis, DCS is generally dosed at 500-1000 mg/day divided twice daily (PDR 1997) with chronic treatment. At a dose of 500 mg/day, blood levels of 25-30 mg/ml are generally maintained. The peak blood levels occur within 3-8 hours after dosing, and it is primarily renally excreted with a half-life of 10 hours. Infrequent side effects in subjects on chronic dosing schedules (who were generally chronically ill with tuberculosis) include drowsiness, headache, confusion, tremor, vertigo, and memory difficulties, paresthesias, and seizure. Side effects correlate well with dosage amount.

Other compounds that may be used in conjunction with the combination therapies of the present invention include pharmacologic agents that increase the level of norepinephrine or acetylcholine in the brain. Pharmacologic agents that increase the level of norepinephrine in the brain include those acting as norepinephrine reuptake inhibitors, for example tomoxetine, reboxetine, duloxetine, venlafaxine (Effexor®), and milnacipran (see, for example, U.S. Pat. No. 6,028,070), and those compounds that cause release of norepinephrine, for example amphetamine, dextroamphetamine (Dexedrine®), pemoline (Cylert®), and methylphenidate (Ritalin®). Pharmacologic agents that increase the level of acetylcholine in the brain include but are not limited to, donepezil HCl or E2020 (Aricept®) and tacrine (THA, Cognex®), which inhibit cholinesterase activity.

The timing of administration and therapeutically effective dose of the particular pharmacologic agent used will depend on the pharmacologic agent, the severity of symptoms, in addition to the age, sex, and size of the subject being treated, among other variables. The particular timing and dose will be selected in order to ensure that a therapeutically effect level of the pharmacologic agent is present in the subject being treated at the time of the extinction training.

In general, the timing of administration of pharmacologic agents according to the present invention will be within about 24 hours, more preferably within about 12 hours, and still more preferably within about 6 hours prior to extinction training. The pharmacologic agent can also be administered within about 12 hours, preferably within about 6 hours, and more preferably within about two hours following extinction training. Accordingly, when a cannabinoid reuptake inhibitor is administered to a subject “in conjunction with” extinction training, the cannabinoid reuptake inhibitor is administered within 24 hours, more preferably within about 12 hours, and still more preferably within about 6 hours prior to extinction training. The cannabinoid reuptake inhibitor can also be administered within about 12 hours, preferably within about 6 hours, and more preferably within about two hours following extinction training.

Where the pharmacologic agent is a cannabinoid reuptake inhibitor, a therapeutically effective dose or amount is that amount of the cannabinoid reuptake inhibitor that enhances levels of endogenous cannabinoid (eCB) reuptake or breakdown in the brain to eCB levels that are higher than without the inhibitor (also referred to as baseline levels). Such an inhibitor would be expected to increase levels of eCB by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% above baseline levels. Methods for measuring eCB levels are well known to those of skill in the art (see, e.g., Freund et al. (2003) Physiol. Rev. 83: 1017-1066 for review). The extent of the increase relative to baseline levels will vary depending on the potency, pharmacokinetics and pharmacodynamics of the particular cannabinoid reuptake inhibitor.

Similarly, where the pharmacologic agent is an agent that enhances NMDA receptor activation or transmission in the brain, a therapeutically effective dose or amount is that amount of the pharmacologic agent that enhances NMDA receptor activation or transmission in the brain to levels that are higher than levels without the agent (also referred to as baseline levels). Such an agent would be expected to enhance NMDA receptor activation or transmission in the brain by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% above baseline levels. Methods of measuring NMDA receptor transmission are well known to those of skill in the art (see, e.g., van Berckel et al. (1997) Neuropsychopharm. 16(5):317-324; Mothet et al. (2000) Proc. Natl. Acad. Sci. USA 97(9): 4926-4931; Boje et al. (1993) Brain Res. 603(2):207-214). When the pharmacologic agent is DCS, the dose will be in the range from 0.2 mg/kg to about 15 mg/kg, preferably between about 0.25 mg/kg and 2.5 mg/kg.

Similarly, when the pharmacologic agent is an agent that increases the level of norepinephrine or acetylcholine in the brain, a therapeutically effective dose or amount is that amount of the pharmacologic agent that increases the level of norepinephrine or acetylcholine in the brain to norepinephrine or acetylcholine levels that are higher than without the agent (also referred to as baseline levels). Such an agent would be expected to increase norepinephrine or acetylcholine levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% above baseline levels. Methods for measuring norepinephrine and acetylcholine levels are well known to those of skill in the art (see, e.g., Preskorn (2004) J. Psychiatr. Pract. 10: 57-63; Frazer (2001) J. Clin. Psychiatry 62: 16-23; Prado et al. (2002) Neurochem. Int. 41: 291-299; Sher et al. (2004) Curr. Top. Med. Chem. 4: 283-297). The amount will vary depending on the potency, pharmacokinetics and pharmacodynamics of a particular agent.

In some embodiments of the invention, the cannabinoid reuptake inhibitor will be administered to the subject on an acute basis in conjunction with extinction training. This method is particularly suitable when the extinction training entails psychotherapy. By administering the cannabinoid reuptake inhibitor on an acute basis, tachyphylaxis is less likely to develop, and therefore less likely to undermine the success of the therapeutic regimen.

The therapeutically effective dose of the pharmacologic agent or agents can be administered using any medically acceptable mode of administration. Although the skilled artisan would contemplate any of the modes of administration known to one of ordinary skill, preferably the pharmacologic agent or agents can be administered according to the recommended mode of administration, for example, the mode of administration listed on the package insert of a commercially available agent.

Extinction Training

The goal of extinction training is to pair a stimulus that previously provoked a deleterious, high-anxiety response with a new learning that the stimulus will not lead to a negative outcome, thereby generating in the subject a new, more appropriate response to the stimulus to replace the previous disproportionate response. In order to accomplish this goal, it is important to ensure that exposure to the previously negative stimulus does not result in an unpleasant outcome for the patient. Accordingly, during extinction training, conditions are modified such that the outcome will be, or is more likely to be, a positive result.

Anxiety Disorders

The methods of the invention contemplate treatment of anxiety disorders by combining (i) administration of a cannabinoid reuptake inhibitor to a subject; and (ii) extinction training provided by any type of psychotherapy that is suitable for the particular anxiety disorder for which the subject is undergoing treatment. Suitable methods of psychotherapy include exposure-based psychotherapy, cognitive psychotherapy, and psychodynamically oriented psychotherapy.

One method of psychotherapy specifically contemplated is the use of virtual reality (VR) exposure therapy to treat an anxiety disorder using the methods of the invention. VR exposure therapy has been used to treat a variety of disorders including anxiety disorders such as the fear of heights (Rothbaum and Hodges (1999) Behav. Modif. 23(4):507-25), as well as specific phobias, eating disorders, and PTSD (Anderson et al. (2001) Bull. Menninger Clin. 65(1):78-91). Because of the prevalence of PTSD in the general population and the successful use of VR therapy to treat PTSD in, for example, Vietnam veterans (Rothbaum et al. (1999) J. Trauma Stress 12(2):263-71) or rape victims (Rothbaum et al. (2001) J. Trauma Stress 14(2):283-93), one embodiment of the present invention specifically contemplates the use of such VR exposure psychotherapy, in conjunction with a cannabinoid reuptake inhibitor, to facilitate extinction of deleterious, high-anxiety responses to neutral stimuli that are associated with PTSD.

Biofeedback

Biofeedback is often aimed at changing habitual reactions to stress that can cause pain or disease. Biofeedback is particularly useful in enabling subjects to learn to control physiological processes that normally occur involuntarily, such as blood pressure, heart rate, muscle tension, and skin temperature.

Many clinicians believe that some of their patients have essentially forgotten how to relax. Feedback of physical responses such as skin temperature and muscle tension provides information that aids subjects in recognizing a relaxed state. For example, one commonly used biofeedback machine detects electrical signals in muscles, and translates these signals into a form that subjects can detect (e.g., flashing bulb, beeper). Subjects can learn to relax tense muscles by learning and repeating behaviors that generate the desirable response from the machine (e.g., reduced beeping, indicative of enhanced relaxation).

The three most common forms of biofeedback therapy are (1) electromyography (EMG), which measures muscle tension, (2) thermal biofeedback, which measures skin temperature, and (3) electroencephalography (EEG, neurofeedback), which measures brain wave activity.

Biofeedback has been demonstrated to be useful, or suggested to be useful, for a range of medical disorders including but not limited to: anorexia nervosa and other eating disorders, anxiety and depression, asthma, autism, back pain, chronic pain, bed-wetting, incontinence, fecal incontinence, constipation, diabetes, sexual disorders, Raynaud's disease, and ADHD.

By administering a cannabinoid reuptake inhibitor in conjunction with extinction training resulting biofeedback therapy, the benefits of the biofeedback therapy can be enhanced. The cannabinoid reuptake inhibitor will enhance consolidation of the response that is learned in biofeedback, thereby reducing the number of biofeedback sessions required to reach the same clinical endpoint and level of benefit to the subject.

Erectile Dysfunction and Sexual Performance

Erectile dysfunction is the inability to obtain and maintain a penile erection sufficient for satisfactory intercourse or other sexual expression. A number of factors can place an individual at risk for this disorder, for example, trauma, pelvic surgery, hypercholesterolemia, ischemic heart disease, peripheral vascular disease, chronic renal failure, diabetes, or the use of medicaments such as antihypertensive medication or digoxin, or illicit drugs, cigarettes or alcohol. Methods for the treatment of erectile dysfunction include but are not limited to: psychotherapy, the use of vacuum devices and penile implants, administration of medicaments such as yohimbine, papaverine and apomorphine, as well as treatment with phosphodiesterase-5 (PDE-5) inhibitors such as vardenafil, tadalafil, and sildenafil.

PDE-5 inhibitors enhance a man's ability to obtain and maintain erections. There are other drugs in clinical trials for treatment of erectile dysfunction that target other physiological pathways. For example, PT-141, from Palatin Technologies, targets the central nervous system. Endothelin antagonists are another class of compounds proposed for treatment of erectile dysfunction. The pharmacological treatments for erectile dysfunction are normally quite effective, but they do not cure the affliction or reverse the underlying problems; rather, they only have an acute, temporary benefit. By administering a cannabinoid reuptake inhibitor to a subject with erectile dysfunction in conjunction with a successful sexual outcome, the heightened confidence and reduced sexual performance anxiety resulting from a successful outcome can be consolidated in the subject's psyche, thereby facilitating extinction of any deleterious performance anxiety associated with sexual intercourse.

Accordingly, one embodiment of the methods of the invention entails administering a cannabinoid reuptake inhibitor to a male in conjunction with extinction training, wherein extinction training comprises:
(1) administration of a pharmacologic agent known to alleviate erectile dysfunction or enhance sexual performance, and
(2) a successful sexual outcome;

While the methods of the invention are useful for patients afflicted with erectile dysfunction, the methods of the invention do not require that the subject be afflicted with erectile dysfunction. In some embodiments of the invention, a pharmacologic agent useful for treating erectile dysfunction is administered to subjects because it improves, or is believed to improve, sexual performance.

In one embodiment of the invention, a subject undergoes a course of treatment ranging from one to ten pharmaceutical interventions comprising:
1. administering to the subject an efficacious PDE-5 inhibitor,
2. a successful sexual outcome, and
3. administering to the subject a cannabinoid reuptake inhibitor.
   At the conclusion of this course of treatment, deleterious performance anxiety in a subject with erectile dysfunction should be substantially eliminated. Therefore and thereafter, the physiological boost (i.e., a PDE-5 inhibitor) required for successful sexual performance is reduced. For erectile dysfunction subjects for whom the etiology is primarily psychogenic, this removal of deleterious performance anxiety may be sufficient to cure the subject, eliminating the need for future pharmaceutical intervention. For erectile dysfunction subjects with significant physiological impediments to achieving or maintaining erections, pharmaceutical therapy may still be required; however, the success rate of that pharmaceutical therapy will be higher, as the physiological boost provided by the drug will no longer have to overcome the additional impediment of negative performance anxiety. In other words, even if it does not provide a cure, the combination of a cannabinoid reuptake inhibitor and one or more PDE-5 inhibitors can improve the efficacy of ongoing treatments by eliminating the negative influence of performance anxiety. Accordingly, the methods and compositions of the invention are useful for the treatment of most erectile dysfunction subjects, not limited to those subjects for whom the affliction is primarily psychogenic.
   Pain

Many individuals suffer from chronic pain, including neuropathic pain. Numerous non-pharmacologic techniques are used to treat chronic pain, including transcutaneous electrical nerve stimulation (TENS), acupuncture, physical therapy, massage, relaxation therapy, biofeedback, and psychotherapy, in addition to pharmacotherapy. Medications from several different drug classes are commonly used to treat chronic pain, including topical agents, tricyclic antidepressants, serotonin specific reuptake inhibitors (SSRIs), anticonvulsants, and nonopioid analgesics.

Recent studies (Science, Vol 303, 1162-1167 (2004)) have demonstrated that people experience pain differently when they believe that the pain will be alleviated. The experience of pain arises from both physiological and psychological factors, including one's beliefs and expectations. Thus, placebo treatments that have no intrinsic pharmacological effects may produce analgesia by altering expectations. In two functional magnetic resonance imaging (fMRI) experiments, researchers found that placebo analgesia was related to decreased brain activity in pain-sensitive brain regions, including the thalamus, insula, and anterior cingulate cortex, and was associated with increased activity during anticipation of pain in the prefrontal cortex, providing evidence that placebos alter the experience of pain.

Given this result, it is clear that a subject's response to painful stimuli is governed by a number of factors, many of which are psychological. If a subject is anxious about the pain, the pain that is experienced in normally worse than if the subject is not anxious about the pain. It is not surprising, therefore, that chronic pain has been treated effectively using cognitive behavioral therapy. The methods of the invention aim to reduce the psychological component associated with chronic pain. One method to do so would be to render permanent a subject's temporal, low-anxiety response to chronic pain. By administering a cannabinoid reuptake inhibitor to a subject in conjunction with extinction training, a beneficial, low-anxiety response to chronic pain can be consolidated and rendered more likely to be repeated in the future.

Insomnia

Extinction training for reducing anxiety associated with insomnia entails subjecting a subject afflicted with the disorder to an environment wherein a stimulus is presented that frequently generates a deleterious, high-anxiety response (i.e., an attempt to fall asleep), but conditions are controlled to reduce anxiety or produce a favorable outcome (i.e., falling asleep relatively easily), or both. According to the methods of the invention, a cannabinoid reuptake inhibitor is administered to a subject within 24 hours, preferably within 4 hours, of extinction training that will reduce anxiety associated with insomnia.

Psychotherapy, biofeedback training, and acupuncture are all non-pharmaceutical methods for treating insomnia. Any of these methods can be combined with a cannabinoid reuptake inhibitor according to the methods of the invention.

Pharmacologic agents useful for treatment of insomnia can be also used in the methods of this invention. Zaleplon, zopiclone, eszopiclone, indiplon, and zolpidem are all central nervous system depressants useful for treatment of insomnia. Benzodiazepines, e.g., lorazepam, clonazepam, oxazepam, flurazepam, triazolam, temazepam, alprazolam, and pharmaceutically acceptable salts thereof, are also frequently used to treat insomnia. By taking a drug that is likely to induce sleep, a subject suffering from insomnia will be more likely to have a positive outcome (i.e., falling asleep), upon exposure to a stimulus (i.e., going to bed and attempting to fall asleep) that often generates substantial anxiety in the subject. The positive outcome, and reduced anxiety, will be reinforced as a new learning by a pharmacologic agent that facilitates extinction such as a cannabinoid reuptake inhibitor. Accordingly, pharmaceutical combinations of (i) a cannibinoid reuptake inhibitor, and (ii) one or more pharmacologic agents useful for treatment of insomnia, are contemplated according to the methods and compositions of the present invention. In some embodiments, subjects can be co-administered a cannabinoid reuptake inhibitor and one or more pharmacologic agents generally known to be useful for treatment of insomnia, including but not limited to zaleplon (between 5 mg and 40 mg), zopiclone (between 2.5 mg and 50 mg), zolpidem (between 2.5 mg and 40 mg), eszopiclone (between 1 mg and 10 mg), indiplon (between about 2.5 mg and 50 mg), triazolam (between about 0.05 mg and 1 mg), clonazepam (between about 0.1 mg and 2 mg), alprazolam (between about 0.1 mg and 2.5 mg), lorazepam (between about 0.5 mg and 2.5 mg) and pharmaceutically acceptable salts thereof.

Animal Training

In some embodiments of the invention, the subject is a mammal other than a human. In some preferred embodiments, the subject is a dog, and a cannabinoid reuptake inhibitor is administered to the dog in conjunction with extinction training. Suitable forms of extinction training include but are not limited to: training to reduce separation anxiety, extinction training to reduce anxiety associated with a particular noise (e.g., thunderstorm), training for obedience skills, and training to reduce destructive behavior.

A subject undergoing treatment with the methods of the invention will experience improved extinction of the deleterious, high-anxiety response that the treatment is intended to eliminate. This facilitated extinction is manifested as reduced anxiety upon exposure to a stimulus that previously prompted the deleterious, high-anxiety response. This reduction in anxiety can leads to improvement in one or more symptoms associated with the various afflictions that can be treated according to the methods of the invention. The efficacy of the methods of the invention can be assessed using any clinically recognized assessment method for measuring a reduction of one or more symptoms of the particular anxiety disorder or other afflictions that are treated. Examples of such assessment methods are described in, for example, Example 11, provided below.

The present invention may be better understood with reference to the following examples. These examples are intended to be representative of specific embodiments of the invention, and are not intended as limiting the scope of the invention.

### EXAMPLES

Examples 1-10 were conducted to examine the effects of cannabinoid reuptake inhibitors, alone or co-administered with pharmacologic agents that enhance NMDA receptor neurotransmission, on conditioned fear extinction. These experiments were conducted using Adult male Sprague-Dawley rats as described in the Materials and Methods section below. Example 11 describes a (prophetic) clinical trial of the effects of cannabinoid reuptake inhibitors, alone or co-administered with pharmacologic agents that enhance NMDA receptor neurotransmission, on augmentation of behavioral exposure therapy for human subjects suffering from a specific phobia.

Materials and Methods for Experiments 1-10:

Animals: Adult male Sprague-Dawley rats (Charles River, Raleigh, N.C., 350-450 g) were used in the present studies. Animals were housed in pairs in a temperature-controlled animal colony, with ad libitum access to food and water, and maintained on a 12 hr light/dark cycle.

In situ hybridization: In situ hybridization was performed as previously described (Ressler et al. (2002) J. Neurosci. 22: 7892-7902). A cDNA clone containing the coding sequence of the rat cannabinoid receptor type 1 (I.M.A.G.E. expressed sequence tag clone, GI Accession # 11375084) was linearized after sequence verification. An antisense riboprobe was generated with T3 RNA polymerase. Slide-mounted sections of snap-frozen rodent brain tissue were post-fixed, proteinase K digested, and blocked followed by overnight hybridization of the tissue at 52° C. with 35S-UTP labeled riboprobes. After a stringent wash protocol, slides were apposed to autoradiography film and hybridization density was qualitatively assessed.

Fear conditioning: All training and testing trials were performed in standardized chambers optimized for the measurement of fear-potentiated startle (see Walker et al. (2002) J. Neurosci. 22: 2343-2351). Animals were pre-exposed to the chambers for 10 min on each of 2 days prior to training for habituation purposes and to minimize the effects of contextual conditioning. On 2 consecutive days following habituation, rats were returned to the same chambers and presented with 10 pairings of a light (3.7 sec) co-terminating with a 0.4-mA, 0.5-sec shock (3.6-min inter-trial interval).

Matching: Twenty-four hours following the last fear-conditioning session, animals were returned to the same chambers and presented with startle stimuli (50-msec, 95-dB white-noise bursts) in the presence or absence of the light conditioned stimulus (light-CS). Increased startle in the presence of the light-CS was taken as a measure of conditioned fear, and the magnitude of the fear response was calculated as the percentage by which startle increased when the light-CS was presented in compound with the startle stimulus versus when it was omitted (fear-potentiated startle or FPS). Using these measurements, animals were divided into groups displaying approximately equal levels of FPS prior to drug treatment and extinction training.

Extinction training: Five days following the last fear conditioning trial, animals were injected intraperitoneally with a test compound or its vehicle in 1 mL/kg volumes and then immediately returned to the same chambers and presented with 15, 30 or 90 presentations of the light-CS in the absence of footshock (3.7-sec light, 30-sec inter-trial interval). One hour following extinction training, a subset of animals was given a short test consisting of startle stimuli in the presence or absence of the light-CS (2-5 light-startle compounds, values shown are averages of all trials). Twenty-four hrs post-extinction training, all animals were tested for the presence of fear-potentiated startle (15 light-startle compounds). As animals showed a large amount of extinction within the testing session (within-session extinction), the FPS values shown for all drug studies are the average FPS during the first five light-startle compounds.

Reinstatement: Animals that had been previously fear-conditioned and extinction-trained were returned to the testing chamber 48 hours and sometimes 96 hours following extinction training and presented with 3 footshocks in the absence of the light-CS (0.4 mA, 0.5 sec shock, 2 min inter-trial interval). Immediately following the unpaired shocks, animals were tested for the presence of fear-potentiated startle (15 light-startle compounds).

Shock Reactivity, Startle, and Activity Measures: Animals that had been previously fear-conditioned were injected with AM404, placed in the training/testing chambers, and presented with 3 unpaired shocks and 42 startle stimuli (0.4-mA, 0.5-sec shocks, 95-dB noise-burst startle). The same group of animals was returned to the same chambers 3 d later, injected with vehicle, and presented with an identical behavioral test. The values shown are the mean integrated voltages of the accelerometers measured over 200-msec periods beginning at the onset of either the shocks or the startle stimuli. Additionally, a measure of spontaneous motor activity was derived from the mean displacement of the accelerometers in the 2 min prior to delivery of the first shock, while animals were exploring the chambers.

Drugs: Rimonabant (SR 141716A, NIMH Drug Supply Program, Bethesda, Md.) and WIN 55,212-2 (Biomol, Plymouth Meeting, Pa.) were dissolved in 100% DMSO. AM404 (Biomol, Plymouth Meeting, Pa.) was dissolved in 70% DMSO, 30% PBS. In experiments in which both rimonabant and AM404 were used, all drugs were dissolved in 100% DMSO. URB-597 (Sigma-Aldrich, St. Louis, Mo.) was dissolved in aqueous DMSO, and dosed at a range between 0.5 mg/kg and 20 mg/kg.

Statistics: Comparisons were made across drug-treatment groups at each test (e.g. 24-hr groups were compared across treatment groups) using ANOVA or Student's t-test with drug or dose as the independent measure, and using Fischer's LSD test for post-hoc analysis.

### Example 1

### In situ Hybridization Study of CB1

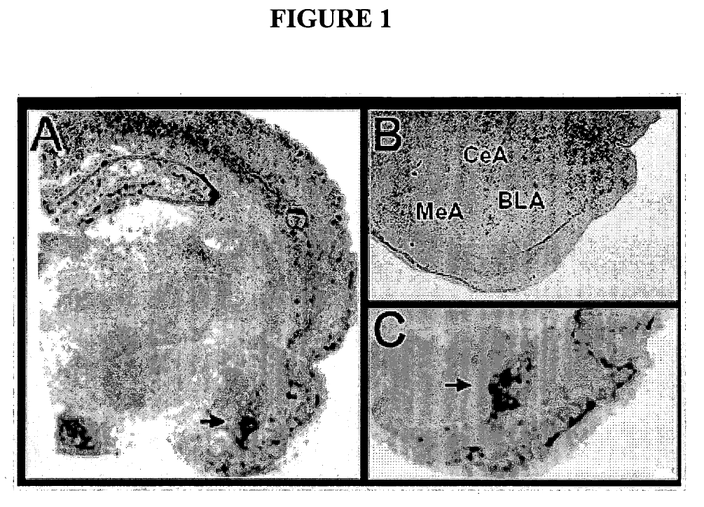

The basolateral amygdala has been repeatedly implicated in the process of extinction of fear with both direct pharmacological inactivation and augmentation studies (Walker et al. (2002) J. Neurosci. 22: 2343-2351; Falls et al. (1992) J. Neurosci. 12(3): 854-863; Davis et al. (2003) Ann. N. Y Acad. Sci. 985, 218-232). In situ hybridization was used to determine if CB1 mRNA was expressed within the rat amygdala and whether it was differentially expressed in the basolateral, medial, and central amygdaloid nuclei. Representative sections from these in situ hybridization studies (FIG. 1), suggest that CB1 mRNA is highly enriched in the BLA, with very little CB1 mRNA expression seen in the central (CeA) or medial nuclei (MeA) of the amygdala. Additionally, the presence of the mRNA for the CB1 protein within the BLA itself suggests that the CB1-mediated signaling taking place in the BLA is part of the intrinsic neurocircuitry of the BLA. These hybridization results are in close agreement with previous studies using immunohistochemical and hybridization techniques (Katona et al. (1999) J. Neurosci. 19(11): 4544-4558; Marsicano & Lutz (1999) Eur. J. Neurosci. 11: 4213-4225). These results indicate that CB1 is enriched in the rat basolateral amygdala.

### Example 2

### Parametric Evaluation of Different Amounts of Extinction Training

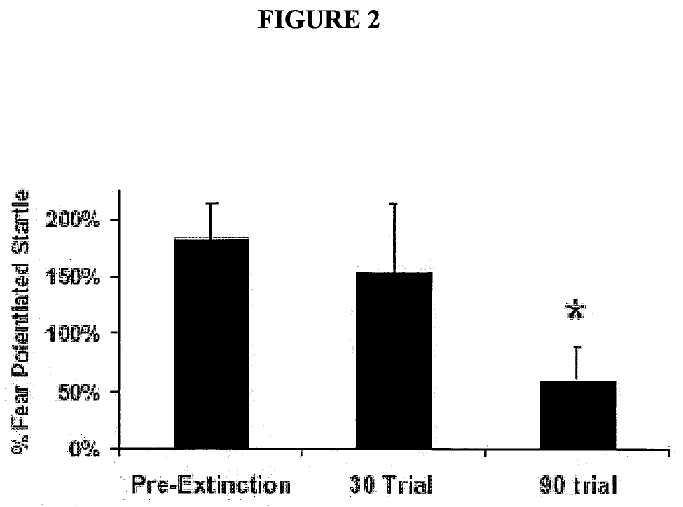

This experiment assessed the effect on fear-potentiated startle of 30 vs. 90 trials of non-reinforced light-conditioned stimulus (CS) presentations. In these studies, animals showed robust fear conditioning prior to extinction-training and varying the number of non-reinforced light-CS presentations decreased the amount of fear animals showed in subsequent testing trials (FIG. 2). In these studies, 90 trials of non-reinforced lights led to significant extinction retention, whereas only 30 trials led to a non-significant reduction in fear (Compared to pre-extinction: 90 trials, F (_{1,27})=4.05, p<0.05; 30 trials, p>0.05).

### Example 3

### Dose-Response Function for the Effect of a CB1 Receptor Antagonist on Extinction

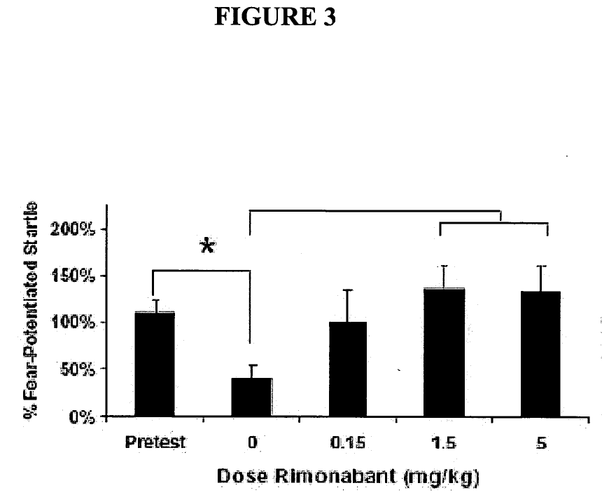

This experiment used the 90-trial extinction protocol described in Example 2 to test the ability of systemic administration of the CB1 antagonist rimonabant on extinction in rats. Acute administration of rimonabant to rats immediately prior to extinction training led to a profound disruption of extinction retention, as evidenced by the fact that rimonabant-treated animals showed significantly higher levels of fear in the presence of the light-CS 24 hrs following extinction training (FIG. 3). This disruption in extinction appeared to be dose-dependent, and animals receiving 1.5 mg/kg or 5 mg/kg of rimonabant showed significantly higher levels of conditioned fear than vehicle-treated controls, and appeared to show virtually no reduction in conditioned fear following extinction training (post-hoc, p<0.01 for 1.5 mg/kg and 5 mg/kg compared to vehicle). The ability of rimonabant to disrupt extinction at the doses used here indicates that the neural process underlying extinction are extremely sensitive to the level of CB1 receptor activation during extinction training.

### Example 4

### Effect of a CB1 Receptor Agonist on Extinction

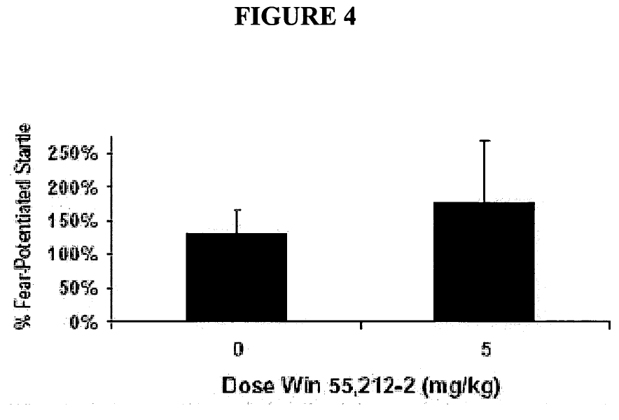

This experiment examined the effect of the CB1 direct agonist WIN 55-212,2 (WIN) on extinction retention. A single dose of WIN (5 mg/kg) was administered prior to a 30 trial-extinction training protocol, to determine if increasing CB1 activation would augment the non-significant reduction in fear observed in Example 2 using this training protocol. However, the administration of 5 mg/kg WIN prior to extinction training did not augment the non-significant reduction in fear observed in Example 2 (FIG. 4). The well-documented emergence of prominent locomotor and analgesic effects following administration of higher doses of WIN (see Herzberg et al. (1997) Neurosci. Lett. 22: 157-160) limited the ability to test the effects of doses of WIN greater than 5 mg/kg.

### Example 5

### Effect of the Cannabinoid Reuptake Inhibitor AM404 on Extinction

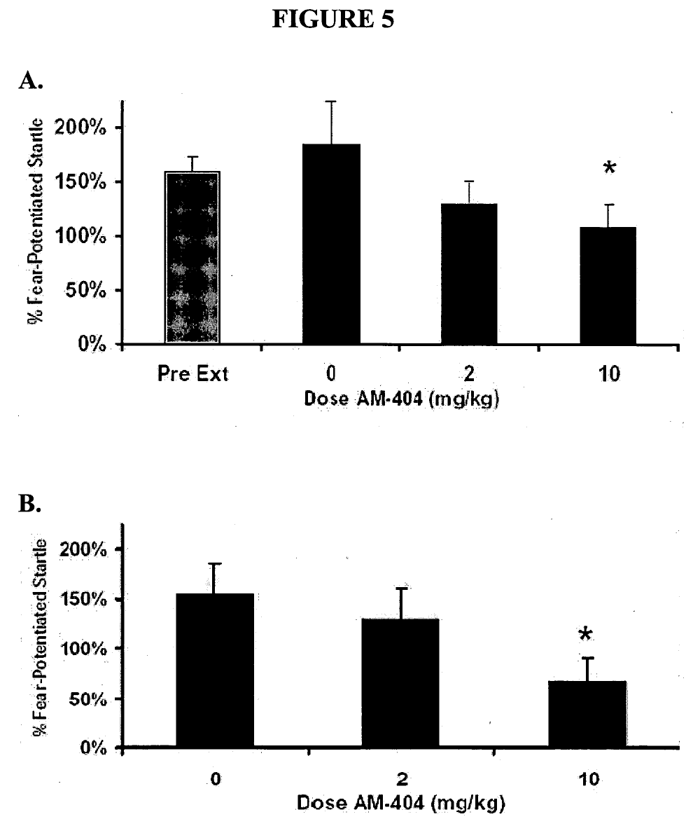

This experiment examined the effect of a cannabinoid reuptake inhibitor, AM404, on extinction. Administration of AM404 prior to 30-trial extinction training led to an enhancement of extinction retention, as AM404 animals showed significantly less fear in the presence of the CS 24 hrs following extinction training (FIG. 5A, main effect of drug treatment F(_{1,70})=4.06, p<0.05). This enhancement of extinction appeared to be dose-dependent, as animals treated with 10 mg/kg AM404 showed less fear than those treated with 2 mg AM404 and significantly less than vehicle-treated animals (10 mg vs. control, post-hoc p<0.05).

A subset of AM404-treated animals were tested 1 hr following extinction, to assess whether the effects of AM404 were likely taking place during the acquisition phase of extinction. The AM404-induced enhancement of extinction was evident 1 hr post extinction, as animals that received the 10-mg/kg dose of AM404 showed significantly less fear than vehicle-treated controls (FIG. 5B, ANOVA linear contrast F(1,37)=4.89, p<0.05; post-hoc comparison, 10 mg/kg vs. vehicle, p<0.05). These findings indicate that AM404 enhances extinction.

### Example 6

### Examination of CB1 Activation in AM404-Enhanced Extinction

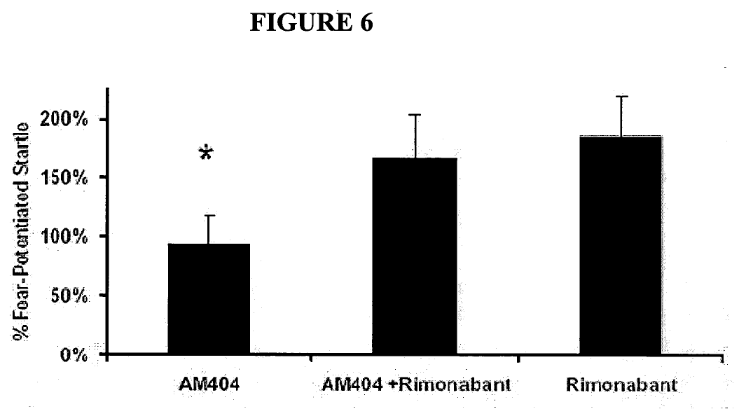

In order to determine whether the AM404-dependent enhancement of extinction requires CB1 activation, animals were divided into three treatment groups. These groups were administered 10 mg/kg AM404, 10 mg/kg AM404+5 mg/kg rimonabant, or 5 mg/kg rimonabant alone, respectively, prior to extinction training (30 trial extinction). Twenty-four hrs post extinction training, animals administered AM404+rimonabant and rimonabant alone showed no decrease in fear potentiated startle (FPS). In contrast, animals treated with 10 mg/kg AM404 alone showed significant extinction relative to animals receiving AM404+rimonabant or rimonabant alone (FIG. 6, F (_{1,23})=5.40, p<0.05, rimonabant and rimonabant+AM404 groups pooled for comparison). Taken together, these results indicate that the enhancement of extinction seen in AM404-treated animals is mediated via CB1 receptor activation.

### Example 7

### Examination of Cue-Exposure and AM404-Enhanced Extinction

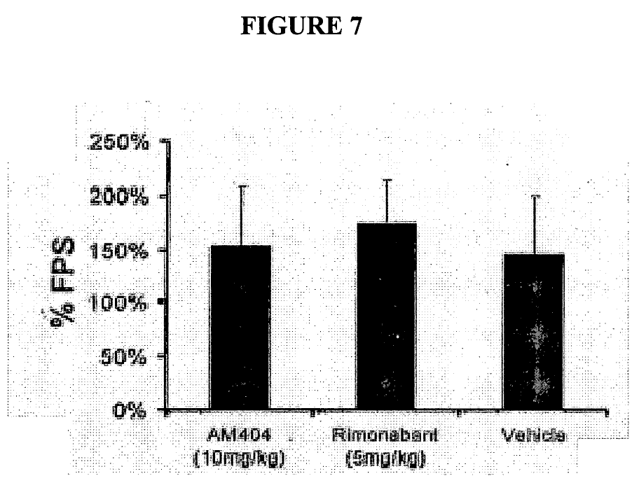

This experiment examined the possibility that AM404 administration itself could lead to decreases in the expression of conditioned fear, even in the absence of cue re-exposure during extinction training. A set of rats was fear-conditioned and matched for equivalent levels of FPS as in the examples described above. On the day on which extinction training was to be performed, animals were administered 10 mg/kg AM404, 5 mg/kg rimonabant, or vehicle, but cue re-exposure was omitted. One hour following drug administration, animals were tested for FPS using a procedure similar to the above studies. The results from these studies indicate that AM404 and rimonabant had no effect on FPS if cue-exposure was omitted, as all drug groups showed similar levels of conditioned fear 1 hr following drug administration (FIG. 7).

### Example 8

### Examination of Analgesic or Locomotor Effects of AM404

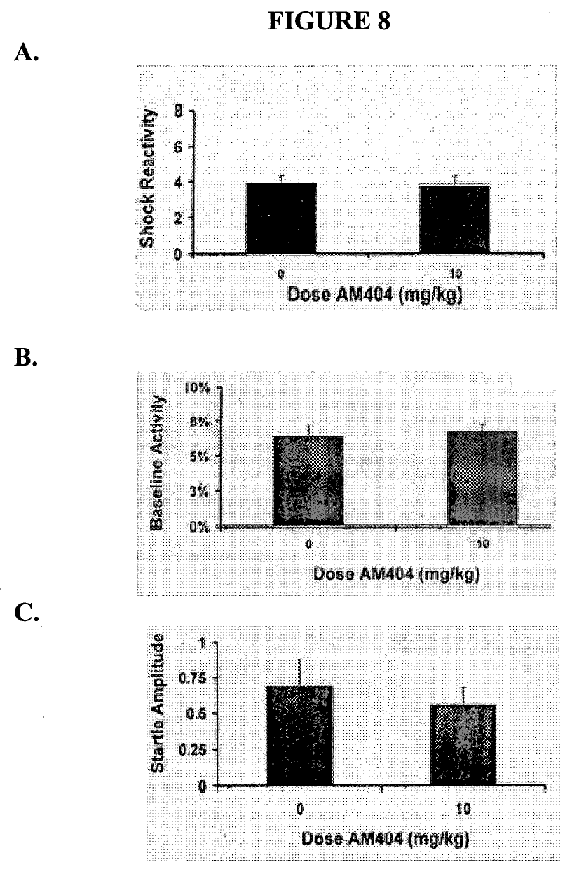

This experiment examined behavioral effects engendered by AM404 treatment. These included the effect of 10 mg/kg AM404 on: 1) shock-reactivity as a measure of pain sensitivity; 2) baseline startle as one measure of anxiety; and 3) general motor activity within the training chambers. Animals were fear-conditioned and then returned to the training chamber several days later and administered 10 mg/kg AM404. Following drug administration, animals were presented with 3 shocks and 42 startle stimuli identical to those used in the above studies. Subsequently, the same animals were returned to the testing chamber 3 days later, injected with vehicle, and similarly tested. The results from these studies (FIGS. 8A-C) showed that administration of AM404 had little effect on shock reactivity or overall locomotor activity levels in the testing chamber (p>0.5 for both comparisons). A non-significant trend toward decreased baseline startle was observed following AM404 administration (p=0.45). Taken together, these results suggest that the administration of AM404 at the doses used in this study are insufficient to generate obvious motor or analgesic effects, and do not affect anxiety levels as measured by baseline startle amplitude.

### Example 9

### Examination of AM404 Treatment on Shock-Induced Reinstatement of Fear

Shock-induced reinstatement was examined 2 days following treatment with AM404 or vehicle during extinction. Previous studies have shown that the level of fear following reinstatement is dependent both on the level of the stressor and the amount of previous extinction, as long as the stressor is delivered in the same context as the original training context (Rescorla & Heth (1975). J. Exp. Psychol. Anim. Behav. Process. 1(1): 88-96; Bouton & King (1983) J. Exp. Psychol. Anim. Behav. Process. 9: 248-265). As animals were matched for equivalent FPS prior to extinction training, the susceptibility of animals to reinstatement can be taken as a secondary measure of the strength of extinction training, and perhaps as a preliminary measure of the resiliency of these inhibitory extinction memories to stressors.

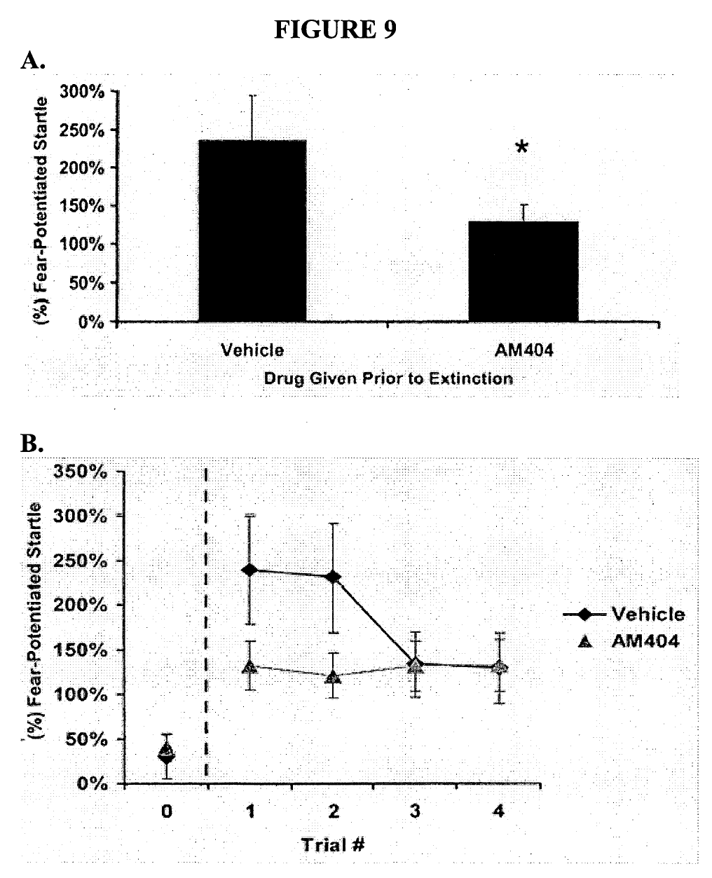

In this experiment, animals that had previously been fear-conditioned, extinction-trained, and tested for extinction retention, were returned to the training chambers and presented with 3 footshocks (in the absence of light-CS presentation) followed by a test for the presence of FPS to the light-CS. During these reinstatement tests, AM404-treated animals showed less reinstatement-induced conditioned fear whereas control animals showed a transient but robust re-emergence of conditioned fear following the unpaired footshocks. This effect was especially prominent during the first two testing trials, where vehicle-treated animals showed significantly more fear to the light CS than their AM404-treated counterparts (FIG. 9A, t(₆₃)=4.5, p<0.05, 2 mg/kg and 10 mg/kg AM404-treated groups pooled for comparison to vehicle). Additionally, examination of within-session extinction demonstrated a significant decrease in FPS among vehicle treated groups, but little change among AM404 treated groups (FIG. 9B, repeated measures ANOVA, Trial X Drug interaction, F (_{1,62})=5.67, p<0.02). Within this period of extinction testing, neither group reached terminal levels of extinction.

### Example 10

### Effect of the Cannabinoid Reuptake Inhibitor URB597 on Extinction

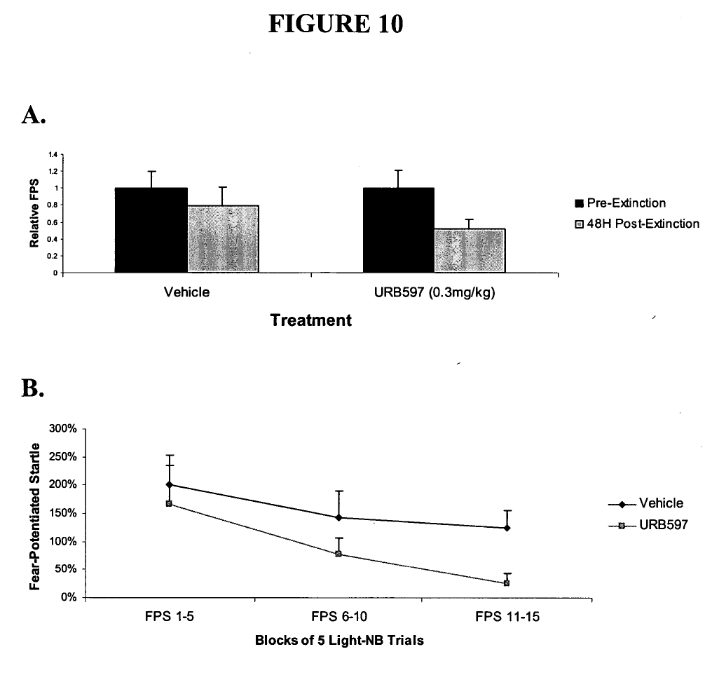

This experiment examined the effect of a second cannabinoid reuptake inhibitor, URB597, on extinction. Animals were trained to be fearful of a 3.7 s second light stimulus, by pairing the presentation of this light with a shock. Animals were then separated into two groups showing approximately equal levels of fear to the light. One of these groups of animals received the fatty-acid amide hydrolase (FAAH) inhibitor URB597 prior to extinction training, while the other group received no drug. Extinction training consisted of 15 presentations of the light without the shocks, prompting the animals to learn that the light no longer predicts the shock. Administration of URB597 prior to 15-trial extinction training led to an enhancement of extinction retention, as URB597 animals showed significantly less fear in the presence of the CS 48 hrs following extinction training (FIG. 10A). Animals that received URB597 showed greater reductions in fear within the testing session, indicating a progressive and enhanced decrease in their levels of fear relative to animals receiving no drug (FIG. 10B). The reductions in conditioned fear observed in URB597-treated animals were retained over several days. At testing sessions conducted 48 and 96 hours after extinction training, animals that did not receive a drug showed no differences in FPS relative to their pre-extinction levels. In contrast, animals that were administered URB-597 exhibited an average decrease in FPS of approximately 30%.

### Discussion for Examples 1-10

These examples demonstrate that: 1) CB1 mRNA is expressed densely and relatively specifically within the rat basolateral amygdala (BLA), a region implicated in the extinction of conditioned fear, and there is little expression seen in the medial and central nuclei; 2) systemic application of a specific CB1 antagonist (SR 141716A) in rats dose-dependently blocks extinction of fear as it does in mice; 3) this dose-dependent blockade of extinction is robust and easily measured using fear-potentiated startle as a measure of fear; 4) systemic application of a cannabinoid reuptake inhibitor such as AM404 or URB597 dose-dependently enhances extinction of fear as measured at different times following cue re-exposure; 5) this enhancement of extinction is not likely due to changes in baseline anxiety, locomotion, or nociception; 6) the enhancement of extinction with AM404 is CB1-dependent; and 7) this enhancement of extinction diminishes reinstatement of fear following footshock.

As indicated earlier, extinction is generally thought to reflect the formation of new inhibitory associations, as opposed to the forgetting of previously formed associations (Pavlov (1927) Conditioned Reflexes (University Press, Oxford); Konorski (1948) Conditioned Reflexes and Neuronal Organization (University Press, London, Cambridge); Bouton and Bolles (1985) Context, Event Memories, and Extinction (Lawrence Erlbaum Associates, Hillsdale, N.J.); Falls and Davis (1995) “Behavioral and Physiological Analysis of Fear Inhibition,” in Neurobiological and Clinical Consequences of Stress: From Normal Adaptation to PTSD, eds. Friedman et al. (Lippincott-Raven Publishers, Philadelphia); Davis et al. (2000) “Neural Systems Involved in Fear Inhibition: Extinction and Conditioned Inhibition,” in Contemporary Issues in Modeling Psychopathology, eds. Myslobodsky and Weiner (Kluwer Academic Publishers, Boston); Rescorla (2001) “Experimental Extinction,” in Handbook of Contemporary Learning Theories, eds. Mowrer and Klein (Erlbaum, Mahwah, N.J.)). Consistent with this view, the evidence to date suggests that the neural mechanisms, neural circuitry, and pharmacology of excitatory fear conditioning and of conditioned fear extinction are similar. For example, systemic administration of the mitogen-activated protein kinase (MAPK) inhibitor, PD98059, as well as intra-amygdala PD98059 infusions, disrupt fear-conditioning as assessed with both freezing (Schafe et al. (2000) J. Neuroscience 20:8177-8187) and shock-motivated avoidance learning (Walz et al. (1999) Behav. Pharmacol. 10:723-730; Walz et al. (2000) Neurobiol. Learn. Mem. 73:11-20) respectively, and intra-amygdala PD98059 infusions also disrupt extinction as assessed with fear-potentiated startle (Lu et al. (2001) J. Neuroscience 21:RC162). As previously noted, intra-amygdala AP5 infusions also block fear conditioning as assessed with either fear-potentiated startle or freezing and also block extinction in these same paradigms (Miserendino et al. (1990) Nature 345:716-718; Falls et al. (1992) J. Neuroscience 12:854-863; Fanselow and Kim (1994) Behav. Neuroscience 108:210-212; Maren et al. (1996) Behav. Neuroscience 110:1365-1374; Lee and Kim (1998) J. Neuroscience 18:8444-8454; Walker and Davis (2000) Behav. Neuroscience 114:1019-1033).

The present findings that animals that had received AM404 during extinction exposure showed less initial fear-potentiated startle when tested following reinstatement is consistent with previous findings in which more extinction training leads to less fear with reinstatement (see Ledgerwood et al. (2004) Behav. Neurosci. 118(3): 505-13). Furthermore, the observed preservation of previous extinction following the presentation of non-reinforced footshocks indicates that the extinction seen following treatment with a cannabinoid reuptake inhibitor is more robust and less susceptible to subsequent stress than the extinction seen in vehicle-treated controls. Collectively, these findings indicate that augmenting eCB-mediated neurotransmission by inhibition of eCB transport or breakdown provide a novel and robust mechanism for enhancing the extinction of fear. Cannabinoid reuptake inhibitors would therefore serve as useful adjuncts in the treatment of anxiety disorders (such as PTSD, panic disorder, and OCD) as well as drug addiction and other disorders that respond to behavioral treatments utilizing extinction processes.

### Example 11

### Clinical Trial of Augmentation of Behavioral Exposure Therapy for Specific Phobia Using Cannabinoid Reuptake Inhibitors

Example 11 outlines a proposed method for demonstrating the effect of cannabinoid reuptake inhibitors, alone or co-administered with a pharmacologic agent that enhances NMDA receptor transmission, combined with psychotherapy. Acrophobia, or fear of heights, has been shown to be responsive to virtual reality exposure (VRE) therapy (Rothbaum et al. (1995) Am. J. Psychiatry 152(4):626-628), and VRE therapy has been well validated for different specific phobias and for post-traumatic stress disorder (Rothbaum et al. (1995) Am. J. Psychiatry 152(4):626-628; Rothbaum et al. (2000) J. Consult. Clin. Psych. 68(6): 1020-1026). With VRE for fear of heights, it was shown that there were significant improvements on all outcome measures for the treated as compared to the untreated groups (Rothbaum et al. (1995) Am. J. Psychiatry 152(4):626-628). Treated participants in this study reported a positive attitude toward treatment, whereas untreated participants reported negative attitudes. VRE treatment for fear of flying demonstrated that VR treatment was equivalent to standard in vivo exposure therapy, both of which showed significant superiority to waitlist control on all outcome measures (Rothbaum et al. (2000) J. Consult. Clin. Psych. 68(6): 1020-1026). In these studies, patients appear to improve steadily across sessions as noted by the decrease in subjective discomfort across sessions as would be expected with incremental habituation or extinction to the fearful stimulus.

In this example, two treatment groups are assessed. In one group, acute treatment with a cannabinoid reuptake inhibitor prior to psychotherapy is used to enhance the effects of VRE therapy. In the second group, both a cannabinoid reuptake inhibitor and a pharmacologic agent that enhances NMDA receptor transmission are administered acutely prior to psychotherapy in order to enhance the effects of VRE therapy. Specifically, acute doses of pharmacologic agents are given to patients shortly before each individual therapy session over 2 weekly sessions to enhance the final level of VRE treatment efficacy. For this example, AM404 is selected as the cannabinoid reuptake inhibitor and DCS is selected as the pharmacologic agent that enhances NMDA receptor transmission.

Dosing Rationale

Cannabinoid reuptake inhibitors and inhibitors of eCB breakdown have not yet been approved for use in humans, and therefore dosing data in humans does not yet exist.

In this proposed example, a therapeutically effective dose of AM404 sufficient to transiently increase endogenous eCB levels is given to a patient acutely prior to psychotherapy for several reasons. The primary reason for this acute dosing strategy, as described more fully below, is to avoid the potential for chronic or daily dosing with cannabinoid reuptake inhibitors to lead to a downregulation of the CB1 receptor, thus interfering with or preventing augmentation of eCB activity (see, e.g., Ressler and Nemeroff (1999) Biol. Psychiatry 46:1219-1233). Furthermore, by giving acute dosing, the emotional inhibitory learning process is only enhanced during the psychotherapy-augmented learning paradigm.

In this example, doses of either 50 mg or 500 mg dose of DCS are administered to subjects on an acute basis prior to psychotherapy. The choice to use AM404 and AM404+DCS in acute treatments, rather than chronic, format is based primarily on two factors. The first factor is the useful clinical benefit that would be gained from a medication used in a time-limited fashion as an adjunct to psychotherapy. The second factor is the possible compensatory changes in CB1 receptor or NMDA receptor levels following chronic administration.

Patient Selection

Although the majority of patients with fear of heights are expected to be simply phobic, it is expected that a substantial minority may be agoraphobic. In this example, a patient must meet DSM-IV criteria for specific phobia, situational type (i.e., fear of heights) or panic disorder with agoraphobia in which heights are the feared stimulus, or agoraphobia without a history of panic disorder, in which heights are the feared stimulus.

Treatment Schedule

In one treatment group, a patient is treated once per week for 2 weeks, with a therapeutically effective dose of AM404 administered only on the day of therapy, approximately 4 hours before the initiation of therapy. Thus a patient receives only two doses of medication or placebo total over the 2-week period.

In a second treatment group, a patient is treated once per week for 2 weeks, with a therapeutically effective dose of AM404 and a 50 mg or 500 mg DCS dose, administered only on the day of therapy, approximately 4 hours before the initiation of therapy. Thus a patient receives only two doses of an AM404+DCS combination or placebo total over the 2-week period.

Virtual reality exposure therapy (VRE) is to a series of footbridges over a canyon, and to a glass elevator that rises 49 floors (Rothbaum et al. (1995) Am. J. Psychiatry 152(4):626-628). During VRE sessions the patient wears a head-mounted display with stereo earphones that provides visual and audio cues consistent with being on a footbridge over a canyon or inside a glass elevator. During therapy, the therapist makes appropriate comments and encourages continued exposure until anxiety has habituated.

During each VRE session, anxiety is rated by subjective units of discomfort (SUDs) on a 0 to 100 scale in which 0 indicates no anxiety and 100 indicates panic-level anxiety. Psychophysiological responses (pulse, BP, GSR) are monitored throughout each exposure session.

Assessment Methods

A patient's response to a therapy session combining VRE and AM404 or VRE and AM404+DCS may be assessed using any of the methods listed below.

a) Interviews

The Initial Screening Questionnaire (Rothbaum et al. (1995) Am. J. Psychiatry 152(4):626-628) is a short screening instrument that is used to screen initial phone inquiries to identify those likely meeting study criteria for fear of heights.

The Structured Clinical Interview for the DSM-IV (Spitzer et al. (1987) Structured Clinical Interview for DSM III-R (SCID) (New York State Psychiatric Institute, Biometrics Research, N.Y.)) is administered to diagnose and screen for various DSM-III-R axis I disorders (e.g., schizophrenia) as well as establish co-morbid diagnoses.

The Clinical Global Improvement (CGI) Scale is a global measure of change in severity of symptoms. The scale is bipolar with 1=very much improved; 7=very much worse; and 4=no change. It has been used extensively in clinical trials for a variety of psychiatric patients (Guy (1976) ECDEU Assessment Manual for Psychotherapy (revised ed., National Institute of Mental Health, Bethesda, Md.)).

b) Self-Report Measures

The Acrophobia Questionnaire (AQ) is a short self-report questionnaire assessing specific symptoms of fear of heights. It is given weekly prior to VRE.

The Attitude Towards Heights Questionnaire (ATHQ) is a separate self-report scale that measures slightly different aspects of avoidance, and other fear of heights related phenomena.

The Rating of Fear Questionnaire (RFQ) (Rothbaum et al. (1995) Am. J. Psychiatry 152(4):626-628) is used to further assess level of fear related to heights in general and the VRE therapy.

The State-Trait Anxiety Inventory (STAI; Spielberger et al. (1970) Manual for the State-Trait Anxiety Inventory (self-evaluation questionnaire) (Consulting Psychologists Press, Palo Alto, Calif.)) is comprised of 40 items divided evenly between state anxiety and trait anxiety. The authors reported reliability for trait anxiety was 0.81; as expected, figures were lower for state anxiety (0.40). Internal consistency ranges between 0.83 and 0.92.

The Beck Depression Inventory (BDI; Beck et al. (1961) Archives of Gen. Psych. 4:561-571) is a 21-item self-report questionnaire assessing numerous symptoms of depression. The authors report excellent split-half reliability (0.93), and correlations with clinician ratings of depression range between 0.62 and 0.66.

c) Therapist Measure

The subjective units of discomfort (SUDs) is scored by the therapist based on the participant's report during the VRE at 5 minute intervals. SUDS are rated on a 0 to 100 scale in which 0 indicates no anxiety and 100 indicates panic-level anxiety

The Behavioral Avoidance Test (BAT) consists of a brief re-exposure to heights via the Virtual Reality environment, in which the therapist assesses the patient's subjective level of fear and avoidance of heights.

d) Psychophysiological Measures

Measurement of heart rate (HR) is performed and stored by a non-invasive, computer controlled monitoring device for assessment of autonomic reactivity during VRE.

Measurement of blood pressure (BP) is performed by a non-invasive, computer controlled sphygmomanometer for assessment of vascular tone and autonomic reactivity during VRE.

Measurement of galvanic skin conductance (GSR) is performed by a non-invasive, computer controlled monitoring device for assessment of autonomic fear responsivity during VRE.

Although the foregoing invention has been described in some detail by way of illustration and example for purposes of clarity of understanding, it will be obvious that certain changes and modifications may be practiced within the scope of the representative embodiments of these concepts presented below.

We claim:

1. A method for facilitating extinction of a deleterious, high-anxiety response in a subject, comprising:
(A) administering to said subject a cannabinoid reuptake inhibitor; and
(B) exposing said subject to extinction training within 24 hours of said administering of said cannabinoid reuptake inhibitor.

2. The method of claim 1, wherein said extinction training is performed within eight hours of said administering of said cannabinoid reuptake inhibitor.

3. The method of claim 1, wherein said extinction training is performed within four hours after said administering of cannabinoid reuptake inhibitor.

4. The method of claim 1, wherein said extinction training is performed within two hours before said administering of cannabinoid reuptake inhibitor.

5. The method of claim 1, wherein said cannabinoid reuptake inhibitor is administered on an acute basis.

6. The method of claim 1, further comprising co-administering to said subject a pharmacologic agent selected from the group consisting of a pharmacologic agent that increases the level of norepinephrine in the brain, a pharmacologic agent that increases the level of acetylcholine in the brain, and a pharmacologic agent that enhances NMDA receptor transmission in the brain.

7. The method of claim 6, wherein said pharmacologic agent comprises DCS, or a pharmaceutically acceptable salt thereof

8. The method of claim 6, wherein said pharmacologic agent increases the level of norepinephrine in the brain, and is selected from the group consisting of amphetamine, dextroamphetamine, pemoline, and methylphenidate.

9. The method of claim 1, wherein said subject is a human.

10. The method of claim 1, wherein said subject is a dog.

11. The method of claim 1, wherein said extinction training comprises psychotherapy.

12. The method of claim 1, wherein said extinction training is selected from the group consisting of exposure-based psychotherapy, cognitive psychotherapy, and psychodynamically oriented psychotherapy.

13. The method of claim 12, wherein said deleterious, high-anxiety response exacerbates symptoms of a medical disorder selected from the group consisting of anxiety disorders, chronic pain, neuropathic pain, insomnia, and erectile dysfunction.

14. The method of claim 13, wherein said medical disorder is an anxiety disorder.

15. The method of claim 14, wherein said anxiety disorder is post-traumatic stress disorder.

16. The method of claim 1, wherein said extinction training comprises biofeedback therapy.

17. The method of claim 1, wherein said extinction training extinguishes a deleterious, high-anxiety response that contributes to a medical disorder selected from the group consisting of anxiety disorders, chronic pain, neuropathic pain, insomnia, and erectile dysfunction.

18. The method of claim 1, wherein said extinction training comprises:
(A) exposing said subject to a stimulus that causes anxiety associated with erectile dysfunction; and
(B) administering to said subject a therapeutically effective dose of a pharmacologic agent, or pharmaceutically acceptable salt thereof, selected from the group consisting of sildenafil, tadalafil, vardenafil, and PT-141.

19. The method of claim 1, wherein said extinction training comprises:
(A) exposing said subject to a stimulus that causes anxiety associated with insomnia; and
(B) administering to said subject a therapeutically effective dose of a pharmacologic agent, or pharmaceutically acceptable salt thereof, selected from the group consisting of eszopiclone, indiplon, zaleplon, zopiclone, zolpidem, lorazepam, clonazepam, oxazepam, flurazepam, triazolam, temazepam, and alprazolam.

20. The method of claim 1, wherein said extinction training comprises:
(A) exposing said subject to a stimulus that causes anxiety associated with a condition selected from the group consisting of chronic pain and neuropathic pain; and
(B) administering to said subject a therapeutically effective dose of a pharmacologic agent, or pharmaceutically acceptable salt thereof, selected from the group consisting of oxycontin, pregabalin, gabapentin, nortriptyline, and amitriptyline.
